SEQUENCE LISTING (3 sequences)

SEQ ID NO: 1 - protein, 3 aa, Artificial
  Synthetic Peptide
  MOD_RES (3)..(3): AMIDATION
PLG

SEQ ID NO: 2 - protein, 3 aa, Artificial
  Synthetic Peptide
PLG

SEQ ID NO: 3 - protein, 4 aa, Artificial
  Synthetic Peptide
  MOD_RES (4)..(4): AMIDATION
YPLG

(19) United States
(12) Patent Application Publication
(10) Pub. No.: US 2008/0004223 A1
(43) Pub. Date: Jan. 3, 2008

(54) DERMATOLOGICAL USES OF THE TRIPEPTIDE MELANOCYTE STIMULATING INHIBITORY FACTOR (MIF)

(76) Inventors: Joseph J. Hlavka, Tuxedo Park, NY (US); Martin F. Schacker, Huntington, NY (US)

Correspondence Address: DIAMOND LAW OFFICE LLC, 1605 JOHN STREET, SUITE 102, FORT LEE, NJ, 07024, US

(21) Appl. No.: 11/747,748

(22) Filed: May 11, 2007

Related U.S. Application Data
(60) Provisional application No. 60/825,070, filed on Sep. 8, 2006.
(60) Provisional application No. 60/801,128, filed on May 17, 2006.

(51) Int. Cl. A61K 38/03; A61P 17/00; A61Q 19/00

(52) U.S. Cl. 514/15; 514/16; 514/17; 514/18

(57) ABSTRACT

The invention provides methods for improving the appearance and/or health of skin using topical compositions that include the tripeptide MIF, which has the chemical formula prolyl-leucyl-glycinamide, and/or related peptides. Additional aspects of the invention provide cosmetic and pharmaceutical compositions for topical application that contain MIF or related peptides.

This application claims the benefit of U.S. provisional patent application Ser. Nos. 60/825,070 filed Sep. 8, 2006 and 60/801,128 filed May 17, 2006, each of which is incorporated by reference herein in its entirety.

### FIELD OF THE INVENTION

The invention relates generally to the field of dermatological treatments for improving the appearance and/or health of skin.

### BACKGROUND OF INVENTION

Melanocyte stimulating inhibitory factor, otherwise known as MIF, MIF-1 or PLG, is a naturally occurring tripeptide having the chemical formula prolyl-leucyl-glycinamide (L-prolyl-L-leucyl-glycinamide; Pro-Leu-Gly-NH₂). The tripeptide MIF is naturally present as the carboxyl terminal tripeptide of oxytocin. MIF has been shown to produce numerous non-endocrine effects on the brain and has been shown to be active in a number of animal models for depression.

MIF was initially isolated and characterized from bovine hypothalmic extracts (Nair, et al., 1971, Biochem. Biophys. Res. Comm. 43(6): 1376-1381) and rat hypothalmic extracts (Celis, et al., 1971, Proc. Natl. Acad. Sci. USA 68(7): 1428-1433). MIF activity was attributed to inhibiting release of melanocyte stimulating hormone, a pituitary hormone known to stimulate melanin production. Neither disclosure suggests or discloses any potential antidepressant activity for MIF.

U.S. Pat. No. 3,708,593 teaches that MIF exhibits antidepressant activity in mice, as shown by a modified Dopa test (Everett, et al., 1966, Proc. 1^{st} Int. Sym. Anti-Depressant Drugs, p. 164).

U.S. Pat. No. 3,795,738 teaches that MIF, alone or in combination with other known drugs, exhibits increased activity against Parkinson's disease.

U.S. Pat. No. 3,931,184 teaches isolation of medicinally pure MIF. A MIF hemihydrate is dissolved in methanol, followed by the addition of diethyl ether, resulting in a white crystalline precipitate of MIF. This pure MIF is collected, washed with ether and dried under vacuum prior to use.

U.S. Pat. No. 4,278,595 teaches that practical use of MIF has been hindered because MIF is rapidly metabolized subsequent to administration and discloses various MIF analogues.

Tyr-MIF-1 (Tyr-Pro-Leu-Gly-NH₂) is a brain derived peptide shown to affect passive avoidance in rats (Hayashi, et al., 1983, Brain Res. Bull. 11: 659-662). Various analogs to Tyr-MIF-1 (i.e., substitutions for the Tyr residue, resulting in Ala-MIF-1, Leu-MIF-1 or Phe-MIF-1) were tested for a possible effect on behavioral and motor activities (Hayashi, et al., 1984, Pharmacology Biochemistry & Behavior 21:809-812). Ala-MIF-1 and Phe-MIF-1, but not Leu-MIF-1, affected passive avoidance behavior in rats. None of these peptides were shown to affect motor behavior.

Kastin, et al. (1984, Pharmacology Biochemistry & Behavior 21: 767-771) discloses that MIF-1 and Tyr-MIF-1 are active as antidepressants. The degree of activity was measured by the water wheel test, a modification of the Porolt swim test.

Kastin, et al. (1985, Pharmacology Biochemistry & Behavior 23: 1045-1049) determined that Tyr-MIF-1 and several Tyr-MIF-1 analogs possess antiopiate activity. Along with Tyr-MIF-1, Phe-MIF-1 was active in inhibiting the analgesic effect of morphine in rats.

Banks, et al. (1986, Am. J. Physiol. 251 [Endocrine Metabolism 14]: E477-E482) identifies the carrier-mediated transport system responsible for delivery of Tyr-MIF-1 to the extracellular brain fluid from the circulatory system.

Petersson et al. (2001, Peptides. 22(9): 1479-84) discloses that oxytocin decreases carrageenan induced inflammation in rats.

Petersson et al. (2004, Physiol. Behav. 83(3): 475-81) discloses that prolyl-leucyl-glycinamide (MIF) shares some effects with oxytocin but decreases oxytocin levels in rats. However, this paper is silent with respect to topical application and effects of MIF.

Pahdy et al. (2005, Mediators Inflamm. (6): 360-5) discloses that oxytocin and melatonin inhibit Calotropis procera latex-induced inflammatory hyperalgesia in rats.

U.S. Pat. No. 6,358,929 discloses the use of a peptide for preventing intolerance reactions of the skin, in particular in cosmetic compositions.

U.S. Pat. No. 6,620,419 discloses cosmetic or dermo-pharmaceutical use of peptides for healing, hydrating and improving skin appearance during natural or induced aging.

U.S. Pat. No. 6,797,697 discloses compositions containing a peptide and a pigment, and the use thereof in darkening the skin.

U.S. Pat. No. 6,809,075 discloses elastin peptide analogs and their use in combination with skin enhancing agents.

U.S. Pat. No. 6,974,799 discloses pharmaceutical, personal care and cosmetic compositions for topical application that contain a tripeptide (Gly-His-Lys) and a tetrapeptide, and which are reportedly useful for treating visible signs of aging.

### SUMMARY OF INVENTION

One embodiment of the invention provides a topical composition that includes: a dermatologically acceptable vehicle; and prolyl-leucyl-glycinamide. The composition may be a pharmaceutical and/or cosmetic composition for the treatment of mammalian skin, such as human skin.

One embodiment of the invention provides a method for the cosmetic treatment of skin that includes the step of: applying a topical composition comprising prolyl-leucyl-glycinamide to a region of skin.

One embodiment of the invention provides a topical pharmaceutical composition that includes: a dermatologically acceptable vehicle; and a pharmaceutically-active amount of prolyl-leucyl-glycinamide.

One embodiment of the invention provides a method for treating an inflammatory skin condition that includes the step of: applying a topical composition comprising prolyl-leucyl-glycinamide to a region of skin in need of treatment for an inflammatory skin condition.

The compositions of the invention may include, in addition to MIF, at least one of a moisturizing agent, a sunscreen agent, an anti-inflammatory agent, a cosmetic agent, a skin-enhancing or conditioning agent and any combination thereof.

One embodiment of the invention provides a method for preparing a topical composition for the treatment of skin, comprising the step of: admixing a dermatologically acceptable vehicle and at least substantially pure prolyl-leucyl-glycinamide [SEQ ID NO: 1].

Peptides including the MIF sequence, Pro-Leu-Glycinamide, may also be used according to the invention instead of MIF itself or in addition to MIF. Such peptides may, for example, be synthetic peptides.

In one embodiment, the peptide is a peptide from three to nine amino acids that includes the carboxyl terminal sequence Pro-Leu-Glycinamide. The peptide may be a synthetic peptide.

In a related embodiment, the peptide is a peptide from three to nine amino acids that includes the sequence Pro-Leu-Gly, such as the tripeptide Pro-Leu-Gly [SEQ ID NO: 2]. In one variation, the sequence Pro-Leu-Gly is present at the amino terminus of the peptide. The peptide may be a synthetic peptide.

Additional features, advantages, and embodiments of the invention may be set forth or apparent from consideration of the following detailed description and claims. Moreover, it is to be understood that both the foregoing summary of the invention and the following detailed description are exemplary and intended to provide further explanation without limiting the scope of the invention as claimed.

### DETAILED DESCRIPTION

The invention provides topical compositions that include the tripeptide MIF and/or related peptides, methods of using the compositions and methods of preparing the compositions.

One embodiment of the invention provides a topical composition that includes: a dermatologically acceptable vehicle; and prolyl-leucyl-glycinamide. The composition may be a pharmaceutical and/or cosmetic composition for the treatment of mammalian skin, such as human skin.

One embodiment of the invention provides a method for the cosmetic treatment of skin that includes the step of: applying a topical composition comprising prolyl-leucyl-glycinamide to a region of skin. A related embodiment provides a method for the cosmetic treatment of skin that includes the step of: applying a topical composition comprising prolyl-leucyl-glycinamide to a region of skin, wherein said application reduces visible signs of aging of at least the region of skin to which the composition is applied. A variation of the embodiments provides for applying the composition on an at least once daily basis. The compositions may, for example, be applied to any of the face, neck and hands.

One embodiment of the invention provides a topical pharmaceutical composition that includes: a dermatologically acceptable vehicle; and a pharmaceutically-active amount of prolyl-leucyl-glycinamide.

One embodiment of the invention provides a method for treating an inflammatory skin condition that includes the step of: applying a topical composition comprising prolyl-leucyl-glycinamide to a region of skin in need of treatment for an inflammatory skin condition. A variation of the embodiment provides for applying the composition on an at least once daily basis.

While not being bound by theory, it is believed that topical application of MIF will reduce sub-pathological and pathological inflammation of, or associated with, the skin, thereby improving the appearance and/or health of the treated skin.

Inflammatory conditions of the skin are prevalent. For example, the prevalence of psoriasis is 1-2% in the general population. Inflammatory conditions of the skin that may be treated with MIF according to the invention include, but are not limited to: psoriasis, eczema, atopic dermatitis, contact (allergic) dermatitis, keloid (hypertrophic scar), lichen simplex chronicus, prurigo nodularis, Reiter syndrome, pityriasis rubra pilaris, pityriasis rosea, stasis dermatitis, rosacea, acne, lichen planus, scleroderma, seborrheic dermatitis, granuloma annulare, dermatomyositis, alopecia areata, lichen planopilaris, vitiligo, and discoid lupus erythematosus.

MIF may be formulated in any type of vehicle (carrier) that is suitable for application and administration to the skin. Without limitation, MIF may, for example, be formulated into a cream (oil-in-water emulsion), lotion, ointment (water-in-oil emulsion), gel, foam, solution and/or suspension, such as those known in the art.

MIF may, for example, be formulated at an effective concentration within the pharmaceutical or cosmetic compositions of the invention in a range of about 0.0001 % to about 90% by weight, or in a range of about 0.05% to about 50% by weight, or in a range of about 0.5% to about 10%, for example about 1.5% by weight. The compositions may also, for example, include MIF at a concentration in the range of 10⁻¹² M (molar) to 10⁻² M, or in a range from 10⁻⁷ M to 10⁻³ M.

### EXAMPLE

The following example illustrates a skin cream (oil-in-water emulsion) embodiment formulation of the invention.

| Component | Weight Amount | |
|---|---|---|
| Mixture (80:20) of cetylstearyl alcohol and | 5.0 | g |
| oxyethylenated cetylstearyl alcohol containing | | |
| 33 moles of oxyethylene | | |
| Glycerol monostearate | 1.5 | g |
| Cetyl alcohol | 0.75 | g |
| Liquid paraffin | 10.0 | g |
| Polydimethylsiloxane | 0.75 | g |
| Glycerin | 4.0 | g |
| Preservatives (optional) | qs | |
| MIF tripeptide | 5.0 | mg |
| Demineralized water | qs 100.0 | g |

Similar creams may, for example, be formulated with from 1 to 1,000 mg of MIF tripeptide.

MIF may also be combined with at least one dermatologically acceptable substance in a topical composition. The CTFA Cosmetic Ingredient Handbook, Ninth Edition (2002) describes a wide variety of non-limiting cosmetic and pharmaceutical ingredients commonly used in the skin care industry, which are suitable for use as additional ingredients in the compositions of the present invention. Additional ingredient classes include, but are not limited to: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantiveness of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents such as moisturizing agents, skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, and vitamins and derivatives thereof.

One embodiment of the invention provides a composition for topical application that includes MIF and at least one different dermatologically active substance, such as a moisturizing agent and/or an anti-inflammatory agent. Moisturizing agents may, for example, be selected from one or more of: occlusive agents; humectant agents; and emollients. Suitable occlusive moisturizing agents imlcude but are not limited to: fatty acids (e.g. lanolin acid); fatty alcohols (e.g. lanolin alcohol); hydrocarbon oils & waxes (e.g. petrolatum); polyhydric alcohols (e.g. propylene glycol); silicone (e.g. dimethicone); sterols (e.g. cholesterol); vegetable & animal fats (e.g. cocoa butter); vegetable waxes (e.g. carnauba wax); and wax esters (e.g. bees wax). Suitable humectant moisturizing agents include but are not limited to: glycerol; honey; urea; alpha-propylene glycol; and alpha-hydroxy acids (and related combinations), such as glycolic acid, lactic acid, malic acid, citric acid, glycolic acid+ammonium glycolate, alpha-hydroxyethanoic acid+ammonium alpha-hydroxyethanoate, alpha-hydroxyoctanoic acid, alpha-hydroxycaprylic acid, hydroxycaprylic acid, mixed fruit acid, tri-alpha hydroxy fruit acids, triple fruit acid, sugar cane extract, L-alpha hydroxy acid. Squalene is an example of a suitable emollient.

Topical anti-inflammatory agents may, for example, include steroids (corticosteroids) such as, but not limited to: hydrocortisone (for example, 0.5-2.5% by weight); clobetasone (such as clobetasone butyrate); triamcinolone (such as triamcinolone acetonide); fluocinolone (such as fluocinolone acetonide); betamethasone valerate; betamethasone dipropionate; diflucortolone (such as diflucortolone valerate); fluticasone (such as fluticasone valerate); hydrocortisone 17-butyrate; mometasone (such as mometasone furoate); methylprednisolone (such as methylprednisolone aceponate); betamethasone dipropionate; and clobetasol (such as clobetasol propionate).

Non-steroidal anti-inflammatory agents useful in the topical composition of the invention include, but are not limited to: the oxicams, such as piroxicam, isoxicam, tenoxicam, sudoxicam, and CP-14, 304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

A further embodiment provides a topical composition that includes MIF and a retinoid, such as tretinoin (all-trans retinoic acid).

Another embodiment of the invention provides a topical composition that includes MIF and a topical sunscreen agent or sunscreen agent combination. Suitable sunscreen agents and combinations thereof include, but are not limited to: aminobenzoic acid, padimate O, and oxybenzone combination; aminobenzoic acid and titanium dioxide combination; avobenzone, octocrylene, octyl salicylate, and oxybenzone combination; avobenzone and octyl methoxycinnamate combination; avobenzone, octyl methoxycinnamate, octyl salicylate, and oxybenzone combination; avobenzone, octyl methoxycinnamate, and oxybenzone combination; dioxybenzone, oxybenzone, and padimate O combination; homosalate; homosalate, menthyl anthranilate, and octyl methoxycinnamate combination; homosalate, menthyl anthranilate, octyl methoxycinnamate, octyl salicylate, and oxybenzone combination; homosalate, octocrylene, octyl methoxycinnamate, and oxybenzone combination; homosalate, octyl methoxycinnamate, octyl salicylate, and oxybenzone combination; homosalate, octyl methoxycinnamate, and oxybenzone combination; homosalate and oxybenzone combination; lisadimate, oxybenzone, and padimate O combination; lisadimate and padimate O combination; menthyl anthranilate; menthyl anthranilate, octocrylene, and octyl methoxycinnamate combination; menthyl anthranilate, octocrylene, octyl methoxycinnamate, and oxybenzone combination; menthyl anthranilate and octyl methoxycinnamate combination; menthyl anthranilate, octyl methoxycinnamate, and octyl salicylate combination; menthyl anthranilate, octyl methoxycinnamate, octyl salicylate, and oxybenzone combination menthyl anthranilate, octyl methoxycinnamate, and oxybenzone combination menthyl anthranilate and titanium dioxide combination; octocrylene and octyl methoxycinnamate combination; octocrylene, octyl methoxycinnamate, octyl salicylate, and oxybenzone combination; octocrylene, octyl methoxycinnamate, octyl salicylate, oxybenzone, and titanium dioxide combination; octocrylene, octyl methoxycinnamate, and oxybenzone combination; octocrylene, octyl methoxycinnamate, oxybenzone, and titanium dioxide combination; octocrylene, octyl methoxycinnamate, and titanium dioxide combination; octyl methoxycinnamate; octyl methoxycinnamate and octyl salicylate combination; octyl methoxycinnamate, octyl salicylate, and oxybenzone combination; octyl methoxycinnamate, octyl salicylate, oxybenzone, and padimate O combination; octyl methoxycinnamate, octyl salicylate, oxybenzone, padimate O, and titanium dioxide combination; octyl methoxycinnamate, octyl salicylate, oxybenzone, phenylbenzimidazole, and titanium dioxide combination; octyl methoxycinnamate, octyl salicylate, oxybenzone, and titanium dioxide combination; octyl methoxycinnamate, octyl salicylate, phenylbenzimidazole, and titanium dioxide combination; octyl methoxycinnamate, octyl salicylate, and titanium dioxide combination; octyl methoxycinnamate and oxybenzone combination; octyl methoxycinnamate, oxybenzone, and padimate O combination; octyl methoxycinnamate, oxybenzone, padimate O, and titanium dioxide combination; octyl methoxycinnamate, oxybenzone, and titanium dioxide combination; octyl methoxycinnamate and padimate O combination; octyl methoxycinnamate and phenylbenzimidazole combination; octyl salicylate; octyl salicylate and padimate O combination; oxybenzone and padimate O combination; oxybenzone and roxadimate combination; padimate O; phenylbenzimidazole; phenylbenzimidazole and sulisobenzone combination; titanium dioxide; titanium dioxide and zinc oxide combination; and trolamine salicylate.

MIF for use in the methods and compositions of the invention may be obtained in any manner. For example, MIF may be isolated or prepared from natural sources or may be prepared by peptide synthesis chemistry, such as by the methods known in the art. Preferably, MIF which is at least substantially pure is used in the preparation of the MIF-containing compositions of the invention. Accordingly, one embodiment of the invention provides a method for preparing a topical composition for the treatment of skin that includes the step of: mixing a dermatologically acceptable vehicle and an at least substantially pure quantity of prolyl-leucyl-glycinamide. At least one other agent such as a moisturizing agent, a sunscreen agent, an anti-inflammatory agent, a cosmetic agent, a skin-enhancing agent and any combination thereof may also be mixed with the dermatologically acceptable vehicle and the at least substantially pure prolyl-leucyl-glycinamide, in the same mixing step or in a different mixing step.

Peptides including an carboxyl-terminal MIF sequence, Pro-Leu-Glycinamide, may also be used according to the invention instead of MIF itself or in addition to MIF. Thus, for any of the embodiments, variations and examples disclosed herein, peptides including the MIF sequence may be used instead of or in addition to MIF itself. Such peptides are preferably obtained in an at least substantially pure form for use in preparing the compositions of the invention and use in the methods of the invention and may, for example, be synthetic peptides.

In one embodiment, the peptide used is a peptide from three to nine amino acids, i.e., a tripeptide, tetrapeptide, peptapeptide, hexapeptide, heptapeptide, octapeptide or nonapeptide, that includes the carboxyl terminal sequence Pro-Leu-Glycinamide. In one variation, the peptide is a peptide from four to nine amino acids in length. For example, the peptide may be Tyr-Pro-Leu-Glycinamide [SEQ ID NO: 3]. In another variation, the peptide is a pentapeptide. The peptide may be a synthetic peptide.

Peptides that include the sequence Pro-Leu-Gly may also be used according to the invention instead of or in combination with MIF and/or any of the peptides disclosed herein. Thus, for any of the embodiments, variations and examples disclosed herein, peptides including the sequence Pro-Leu-Gly may be used instead of or in addition to MIF itself. Peptides that include the sequence Pro-Leu-Gly are preferably obtained in at least substantially pure form for use in the compositions and methods of the invention and may, for example, be synthetic peptides.

In one embodiment, the peptide is a peptide from three to nine amino acids in length that includes the sequence Pro-Leu-Gly. In one variation, the sequence Pro-Leu-Gly is present at the carboxyl terminus of the peptide. In one variation, the sequence Pro-Leu-Gly is present at the amino terminus of the peptide. In one variation, the peptide is a peptide from four to nine amino acids, i.e., a tetrapeptide, peptapeptide, hexapeptide, heptapeptide, octapeptide or nonapeptide. In one variation, the peptide is a pentapeptide. The peptide may be a synthetic peptide.

Dermatologically and/or pharmaceutically acceptable salts of any of the peptides of this disclosure may also be used according to the invention.

In embodiments of the invention in which additional amino acids over the core peptide sequences are present, the additional amino acids may include without limitation the known naturally occurring amino acids (typically referred to by both their common three letter abbreviation and single letter abbreviation), stereoisomers, such as the D-forms, modifications of naturally occurring amino acids and -amide forms of amino acids. The term “carboxyl terminal sequence” as used herein means the sequence of a peptide of the invention occurring at the carboxy-terminus end of the peptide with respect to conventional peptide nomenclature irrespective of whether the particular peptide terminates with a carboxyl group.

Each of the patents, patent applications and other documents cited herein is hereby expressly incorporated by reference in it entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A topical dermatological composition comprising:
a dermatologically acceptable vehicle; and
a synthetic peptide having prolyl-leucyl-glycinamide as its carboxyl terminal sequence or a salt thereof.

2. The composition of claim 1, wherein the synthetic peptide is three to nine amino acids in length.

3. The composition of claim 2, wherein the synthetic peptide is a pentapeptide.

4. The composition of claim 1, wherein the composition further comprises at least one skin conditioning agent.

5. The composition of claim 4, wherein the at least one skin conditioning agent comprises at least one moisturizing agent.

6. The composition of claim 1, further comprising at least one topical anti-inflammatory agent.

7. The composition of claim 1, wherein the synthetic peptide is prolyl-leucyl-glycinamide.

8. The composition of claim 7, wherein the composition further comprises at least one skin conditioning agent.

9. The composition of claim 8, wherein the at least one skin conditioning agent comprises at least one moisturizing agent.

10. The composition of claim 7, further comprising at least one topical anti-inflammatory agent.

11. A method for the cosmetic treatment of skin, comprising the step of:
applying a topical dermatological composition according to claim 1 to a region of skin.

12. A method for the cosmetic treatment of skin, comprising the step of:
applying a topical dermatological composition according to claim 2 to a region of skin.

13. A method for the cosmetic treatment of skin, comprising the step of:
applying a topical dermatological composition according to claim 3 to a region of skin.

14. A method for the cosmetic treatment of skin, comprising the step of:
applying a topical dermatological composition according to claim 7 to a region of skin.

15. A method for manufacturing a topical dermatological composition, comprising the step of:
admixing a dermatologically acceptable vehicle and an at least substantially pure amount of a peptide having prolyl-leucyl-glycinamide as its carboxyl terminal sequence or a salt thereof.

16. The method of claim 15, wherein the peptide is three to nine amino acids in length.

17. The method of claim 16, wherein the peptide is a pentapeptide.

18. The method of claim 15, wherein the peptide is prolyl-leucyl-glycinamide.

19. The method of claim 15, wherein the peptide is synthetic.

20. The method of claim 15, further comprising admixing at least one skin conditioning agent.

21. A topical dermatological composition comprising:
a dermatologically acceptable vehicle; and
a synthetic peptide of three to nine amino acids comprising the sequence Pro-Leu-Gly or a salt thereof.

22. The composition of claim 21, wherein the carboxyl terminal sequence of the peptide is Pro-Leu-Gly.

23. A method for the cosmetic treatment of skin, comprising the step of:
applying a topical dermatological composition according to claim 21 to a region of skin.

24. A method for the cosmetic treatment of skin, comprising the step of:
applying a topical dermatological composition according to claim 22 to a region of skin.
